(19) United States
(12) Patent Application Publication
(10) Pub. No.: US 2025/0195799 A1
(43) Pub. Date: Jun. 19, 2025

(54) INHALER

(71) Applicant: KT&G CORPORATION, Daejeon (KR)

(72) Inventors: Tae Heon KIM, Daejeon (KR); Jae Hyun KIM, Daejeon (KR); Mi Jeong LEE, Daejeon (KR); Minseok JEONG, Daejeon (KR); Yongmi JUNG, Daejeon (KR); Eunmi JEOUNG, Daejeon (KR); Tae Young CHUNG, Daejeon (KR); Seung Kyu HAN, Daejeon (KR)

(73) Assignee: KT&G CORPORATION, Daejeon (KR)

(21) Appl. No.: 18/286,426

(22) PCT Filed: May 31, 2023
(86) PCT No.: PCT/KR2023/007445

§ 371 Date: Feb. 26, 2025

(30) Foreign Application Priority Data
Jul. 1, 2022 (KR) 10-2022-0081055

(51) Int. Cl. A61M 15/00

(52) U.S. Cl. CPC A61M 15/009; A61M 15/0001; A61M 2202/0007; A61M 2202/04; A61M 2205/12

(57) ABSTRACT

An inhaler according to one embodiment may comprise: a housing having one surface, the other surface opposite the one surface, and a plurality of side surfaces connecting the one surface and the other surface; a reservoir located inside the housing and storing an inhalable composition; a canister accommodating an inhalable composition to be stored in the reservoir and openably/closably connected to the reservoir; a relief valve spaced apart from the canister and installed to open and close the reservoir with respect to the outside; and a lever that controls the opening/closing operation of at least one of the canister or the relief valve, wherein the lever can control the canister to be opened after the relief valve is opened.

### TECHNICAL FIELD

Disclosed is an inhaler.

### BACKGROUND ART

In general, an inhaler is a device used to inhale a composition such as medication through the oral cavity or nasal cavity as a liquid or gas in the process of inhalation. Such an inhaler may include a container accommodating an inhalable composition, and the composition may be sprayed from the container through a thin tube to the oral cavity or nasal cavity through an intake to be inhaled by a user.

The above description has been possessed or acquired by the inventor(s) in the course of conceiving the present disclosure and is not necessarily an art publicly known before the present application is filed.

Prior Document: Korean Patent Gazette No. 10-2021229 (Published on Sep. 11, 2019)

### DISCLOSURE OF THE INVENTION

### Technical Goals

An object according to an embodiment is to provide an inhaler capable of performing smooth filling by discharging a residual gas in a reservoir before filling the reservoir with an inhalable composition.

The technical tasks obtainable from the present disclosure are non-limited by the above-mentioned technical tasks. And, other unmentioned technical tasks can be clearly understood from the following description by those having ordinary skill in the technical field to which the present disclosure pertains.

### Technical Solutions

An inhaler according to an embodiment for achieving the above objects includes: a housing having one surface, the other surface opposite to the one surface, and a plurality of side surfaces connecting the one surface and the other surface; a reservoir which is disposed inside the housing and stores an inhalable composition; a canister which accommodates the inhalable composition to be stored in the reservoir and is connected to the reservoir to be opened or closed; a relief valve which is disposed to be spaced apart from the canister and provided to open and close the reservoir to the outside; and a lever which controls an opening and closing operation of at least one of the canister or the relief valve. The lever controls the canister to be opened after the relief valve is opened.

According to an aspect, the inhaler may further include a relief bar having one end coupled to the relief valve and extending toward the lever. The relief bar may be positioned between the lever and the canister.

According to an aspect, the lever may further include a support member which extends in a first direction toward the canister; and a protrusion member having an end portion rotatably coupled to the support member around a rotation shaft provided in the support member, and the other end of the relief bar may be positioned between the protrusion member and the other surface of the housing.

According to an aspect, when the lever is pressed, the protrusion member may move the relief bar in a second direction toward the one surface of the housing while being moved in the first direction.

According to an aspect, when the lever is pressed in the first direction, the support member may restrict rotation of the protrusion member, and when the lever returns to an original position, the support member may allow the rotation of the protrusion member.

According to an aspect, the protrusion member may be disposed so that rotation in a direction toward the canister is not restrained and rotation in a direction away from the canister is restrained in a state where the end portion stands toward the one surface of the housing.

According to an aspect, when the canister is opened, the protrusion member may be positioned between the relief bar and the canister.

According to an aspect, the lever may further include a wedge member extending toward a bottom surface of the canister, and a distance between the wedge member and the bottom surface of the canister may be greater than a distance between the protrusion member and the relief bar.

According to an aspect, the wedge member may include an inclined surface inclined to be adjacent to the bottom surface along the first direction, and when the lever is further pressed in the first direction in a state where the inclined surface comes into contact with the bottom surface, the inclined surface may move the canister in the second direction toward the one surface of the housing.

According to an aspect, when the lever reaches a first position in which the relief valve is opened, the inhalable composition remaining in the reservoir may be discharged to the outside, and when the lever reaches a second position in which one side of the canister is opened, the reservoir may be filled with the inhalable composition accommodated in the canister.

According to an aspect, the relief bar may open the relief valve when the relief bar moves in a second direction toward the one surface of the housing, and close the relief valve when the relief bar returns to an original position.

According to an aspect, the canister may include: an accommodation portion which accommodates the inhalable composition; and an injection hole having one side connected to the reservoir and the other side positioned in the accommodation portion, and the injection hole may be opened or closed to allow or restrict movement of the inhalable composition to the reservoir.

According to an aspect, the injection hole may be opened when the accommodation portion moves in a second direction from the other surface to the one surface of the housing, and may be closed when the accommodation portion returns to an original position.

### Effects

According to the inhaler according to an embodiment, there is an effect of providing smooth filling by discharging the residual gas in the reservoir before filling the reservoir with the inhalable composition.

The effects of the inhaler are not limited to the above-mentioned effects, and other unmentioned effects can be clearly understood from the following description by one of ordinary skill in the art to which the present disclosure pertains.

### BRIEF DESCRIPTION OF DRAWINGS

FIG. 1 is a perspective view of an inhaler according to an embodiment.

FIG. 2 is a cross-sectional view of an inhaler according to an embodiment.

FIG. 3 illustrates a relief valve that operates in association with a lever.

FIG. 4 illustrates a two-step operation process of a lever.

FIG. 5 illustrates an opening or closing operation of a relief valve and a canister when a lever operates in the same order as in FIG. 4.

The accompanying drawings illustrate desired embodiments of the present disclosure and are provided together with the detailed description for better understanding of the technical idea of the present disclosure. Therefore, the present disclosure should not be construed as being limited to the embodiments set forth in the drawings.

### BEST MODE FOR CARRYING OUT THE INVENTION

Hereinafter, embodiments will be described in detail with reference to the illustrative drawings. Regarding the reference numerals assigned to the components in the drawings, it should be noted that the same components will be designated by the same reference numerals, wherever possible, even though they are shown in different drawings. Further, in the following description of the present embodiments, a detailed description of publicly known configurations or functions incorporated herein will be omitted when it is determined that the detailed description obscures the subject matters of the present embodiments.

In addition, the terms first, second, A, B, (a), and (b) may be used to describe constituent elements of the embodiments. These terms are used only for the purpose of discriminating one constituent element from another constituent element, and the nature, the sequences, or the orders of the constituent elements are not limited by the terms. When one constituent element is described as being “connected”, “coupled”, or “attached” to another constituent element, it should be understood that one constituent element can be connected or attached directly to another constituent element, and an intervening constituent element can also be “connected”, “coupled”, or “attached” to the constituent elements.

The same name may be used to describe an element included in the embodiments described above and an element having a common function. Unless otherwise mentioned, the descriptions on the embodiments may be applicable to the following embodiments and thus, duplicated descriptions will be omitted for conciseness.

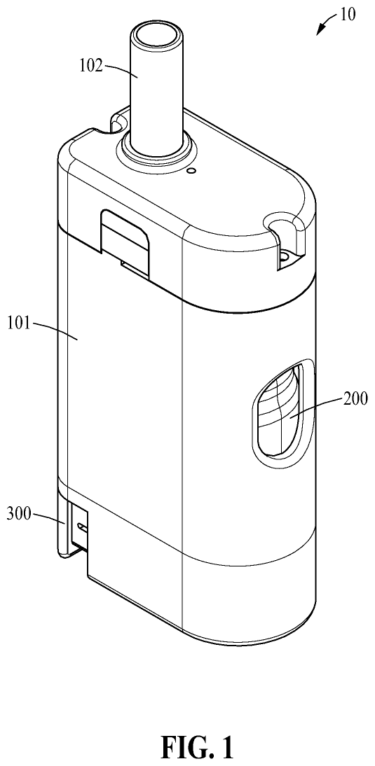

FIG. 1 is a perspective view of an inhaler 10 according to an embodiment.

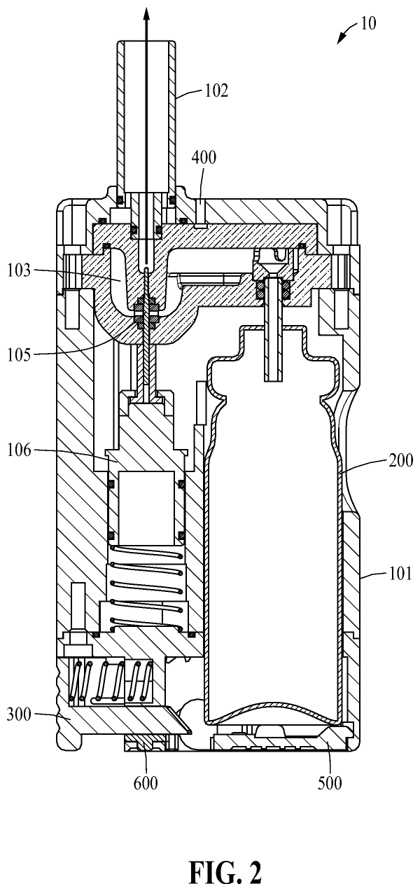

FIG. 2 is a cross-sectional view of the inhaler 10 according to an embodiment.

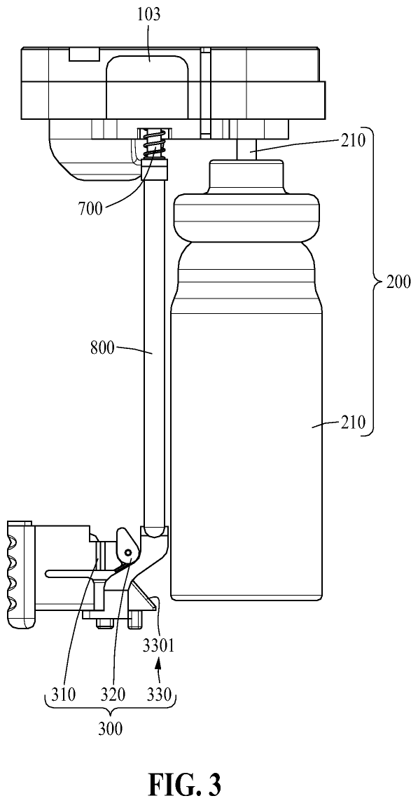

FIG. 3 illustrates a relief valve 700 that operates in association with a lever 300.

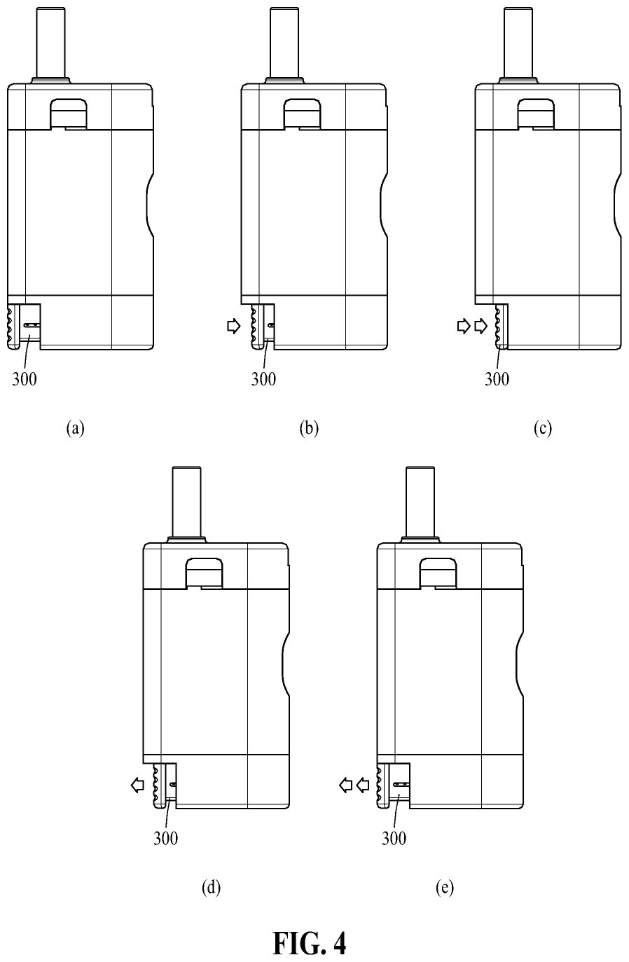

FIG. 4 illustrates a two-step operation process of the lever 300.

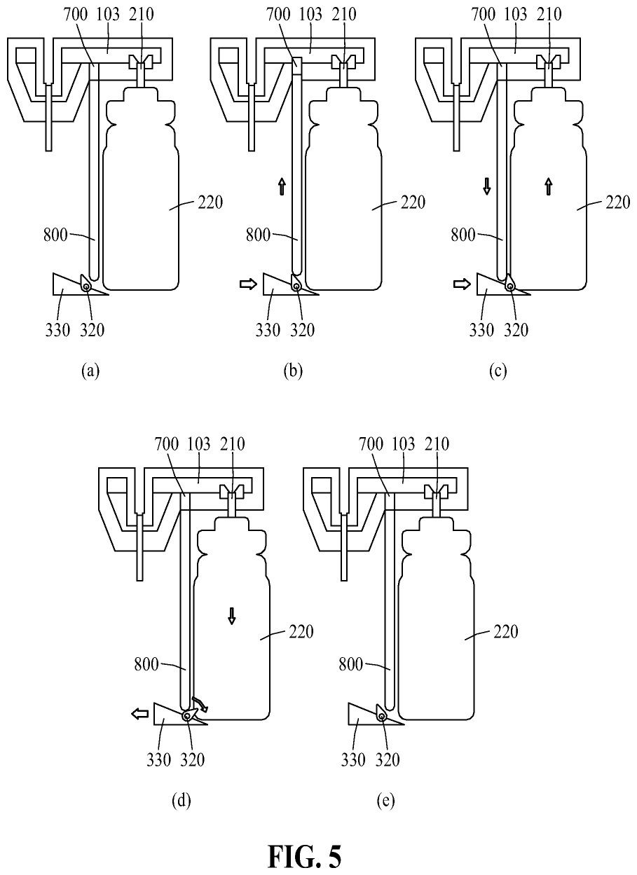

FIG. 5 sequentially illustrates an opening or closing operation of the relief valve 700 and a canister 200 when the lever 300 operates in the same order as in FIG. 4.

Referring to FIG. 1, the inhaler 10 according to an embodiment may include a housing 101, a mouthpiece 102, a reservoir 103, the canister 200, and the lever 300.

The housing 101 may include a first surface formed on one surface, a second surface opposite to the first surface, and a plurality of side surfaces connecting the first surface and the second surface. The first surface of the housing 101 may be, for example, a surface located on the top of the housing 101, and the second surface may be, for example, a bottom surface of the housing 101.

The mouthpiece 102 may be disposed on the first surface of the housing 101. A user may inhale an inhalable composition accommodated in the inhaler 10 through the mouthpiece 102. At this time, the user may inhale the composition, for example, in the form of an aerosol or in the form of a powder. Hereinafter, the inhaler 10 according to an embodiment will be described using the inhaler 10 that sprays an inhalable 10 composition in the form of an aerosol as an example.

Referring to FIGS. 1 and 2, a reservoir 103 may be disposed inside the housing 101. Since one side of the canister 200 is connected to the reservoir 103, the inhalable composition accommodated in the canister 200 may be moved to and stored in the reservoir 103. In addition, as the user applies a suction force through the mouthpiece 102, the inhalable composition stored in the reservoir 103 may be discharged through the mouthpiece 102 in the form of an aerosol, and accordingly, the inhalable composition stored in the reservoir 103 may be gradually consumed.

The canister 200 may be mounted inside the housing 101. In this case, the canister 200 may be mounted interchangeably inside the housing 101. In addition, the canister 200 may accommodate an inhalable composition therein. A certain amount of the composition of the canister 200 may be filled in the reservoir 103.

The lever 300 may be pressed by the user to fill the inside of the reservoir 103 with the composition accommodated in the canister 200. The lever 300 may form one side surface of the housing 101 and may be positioned adjacent to the second surface. When the user presses the lever 300, the lever 300 may push up a bottom surface of the canister 200 so that an injection hole 220 of the canister 200 communicates with the reservoir 103, and the composition may move from the canister 200 to the reservoir 103 through the injection hole 220.

Hereinafter, a direction from one side surface of the housing 101 where the lever 300 is located to the canister 200 is defined as a first direction, and a direction from the second surface of the housing 101 to the first surface is defined as a second direction.

Meanwhile, the relief valve 700 may be provided on the reservoir 103, and a relief vent hole 400 may be provided on the first surface of the housing 101. The relief valve 700 may be interlocked with the lever 300 through a relief bar 800, a relief bar movement protrusion 320, or the like, and the relief valve 700 may operate to open before the lever 300 pushes the canister 200 up. Accordingly, a residual gas in the reservoir 103 may be discharged before the reservoir 103 is filled with the inhalable composition.

The relief valve 700 will be described in more detail with reference to FIGS. 3 to 5 below.

Referring to FIG. 2, in the inhaler 10 according to an embodiment, the user may visually identify a remaining amount of the composition stored in the reservoir 103 through a viewing window (not shown) provided on the housing 101. In addition, the inhaler 10 according to an embodiment may include the counter (not shown) that counts the number of times of filling in connection with a vertical movement of the canister 200 during the filling of the reservoir 103, and a display window (not shown) that displays a remaining amount of the composition in the canister 200. The composition stored in the reservoir 103 may be sprayed in the form of an aerosol as the user applies a suction force to the mouthpiece 102. At this time, an inhalation interlocking valve 105 that opens and closes the reservoir 103 by the suction force may operate, and the opening and closing operation of the inhalation interlocking valve 105 may be controlled by a piston 106. The inhaler 10 according to an embodiment may further include a cover 500 and a locking device 600 for preventing the canister 200 from being separated.

As described above, the inhaler 10 according to an embodiment uses a method of filling the reservoir 103 with the inhalable composition accommodated in the canister 200, and thus, if a residual gas remains in the reservoir 103 during repeated refilling, it may be not easy to fill the reservoir 103 with the composition of the canister 200. Accordingly, the relief valve 700 interlocked with the filling lever 300 may be applied to the inhaler 10 according to an embodiment to facilitate the filling of the reservoir 103.

Referring to FIG. 3, the relief valve 700 of the inhaler 10 according to an embodiment may be provided on the reservoir 103 to be spaced apart from the one side of the canister 200. The relief valve 700 may be opened or closed to allow the reservoir 103 to communicate with the outside or block the reservoir 103.

The opening or closing operation of the relief valve 700 may be controlled by the lever 300 described above. For example, when the lever 300 is pressed by the user, the lever 300 may control the relief valve 700 so that the relief valve 700 is opened first and then the canister 200 is opened.

As described above, as the relief valve 700 interlocked with the lever 300 is applied to the inhaler 10 according to an embodiment, the residual gas in the reservoir 103 may be discharged and the reservoir 103 may be smoothly filled with the inhalable composition from the canister 200.

The lever 300 may be pressed by the user. At this time, the lever 300 may move toward the canister 200 to reach a first position, and when the lever 300 is further pressed by the user, the lever 300 may reach the second position. When the lever 300 is in the first position, the relief valve 700 may be opened, and when the lever 300 is in the second position, the relief valve 700 may be closed and the injection hole 220 of the canister 200 may be opened.

Specifically, the relief valve 700 may be interlocked with the lever 300 through the relief bar 800 and may be opened or closed by raising or lowering of the relief bar 800. The relief bar 800 may be disposed between the lever 300 and the canister 200. The relief bar 800 may have one end coupled to the relief valve 700 and the other end extending toward the lever 300. The relief bar 800 may open the relief valve 700 as the relief bar 800 moves in the second direction, and may close the relief valve 700 as the relief bar 800 returns to its original position.

The lever 300 may include a support member 310 and a protrusion member 320.

The support member 310 may extend toward the canister 200.

The protrusion member 320 may be rotatably coupled to the support member 310 around a rotation shaft provided in the support member 310. The protrusion member 320 may have an end portion protruding in a radial direction from the rotation shaft at one side. The end portion of the protrusion member 320 may be disposed in a standing state toward the first surface of the housing 101.

The other end of the relief bar 800 may be positioned between the protrusion member 320 and the second surface of the housing 101. In addition, the end portion of the protrusion member 320 may be inclined in a direction away from the canister 200 with respect to the rotation shaft.

When the lever 300 is pressed in the first direction, the support member 310 may restrain the rotation of the protrusion member 320. That is, the rotation of the protrusion member 320 in a direction away from the canister 200 may be restrained in a state where the end portion stands toward the first surface of the housing 101.

Therefore, when the lever 300 is pressed, the protrusion member 320 also moves in the first direction to reach the first position, and at this time, the protrusion member 320 may come into contact with the relief bar 800 and push the relief bar 800 up in the second direction without rotating.

As a result, when the lever 300 reaches the first position, the inhalable composition remaining in the reservoir 103 may be discharged to the outside.

Meanwhile, when the lever 300 returns to its original position, the support member 310 may allow the rotation of the protrusion member 320. That is, the rotation of the protrusion member 320 in the direction toward the canister 200 may not be restrained in a state where the end portion stands up toward the first surface of the housing 101.

Accordingly, when the lever 300 moves from the second position or a position beyond the first position in a direction opposite to the first direction, the protrusion member 320 may pass through the relief bar 800 and return to its original position without pushing the relief bar 800 up.

Referring back to FIG. 3, the lever 300 may further include a wedge member 330 extending toward the bottom surface of the canister 200.

The wedge member 330 may include an inclined surface 3301 inclined to be more adjacent to the bottom surface of the canister 200 along the first direction.

Accordingly, when the lever 300 is further pressed in the first direction while the inclined surface 3301 is in contact with the bottom surface, the inclined surface 3301 may move the canister 200 in the second direction.

A distance between the inclined surface 3301 of the wedge member 330 and the bottom surface of the canister 200 may be greater than a distance between the protrusion member 320 and the relief bar 800. Accordingly, when the lever 300 is moved in the first direction, the protrusion member 320 may first come into contact with the other end of the relief bar 800 before the inclined surface 3301 comes into contact with the bottom surface of the canister 200. That is, the relief valve 700 provided on the reservoir 103 may be opened first, immediately before the lever 300 pushes the canister 200 to fill the reservoir 103.

Meanwhile, when the canister 200 is opened, that is, when the lever 300 reaches the second position and the inclined surface 3301 pushes the canister 200 up in the second direction, the protrusion member 320 may be positioned between the relief bar 800 and the canister 200.

Eventually, when the lever 300 reaches the second position, the reservoir 103 may be filled with the inhalable composition accommodated in the canister 200.

Referring back to FIG. 3, the canister 200 of the inhaler 10 according to an embodiment may include an accommodation portion 210 and the injection hole 220.

The accommodation portion 210 may accommodate an inhalable composition.

One side of the injection hole 220 may be connected to the reservoir 103 and the other side thereof may be positioned in the accommodation portion 210. The injection hole 220 may operate to be opened or closed with respect to the reservoir 103. For example, when the lever 300 is in the second position, the injection hole 220 may be opened. When the injection hole 220 is opened, the inhalable composition accommodated in the accommodation portion 210 may be allowed to move to the reservoir 103. On the other hand, when the lever 300 is not in the second position, the injection hole 220 may be closed. When the injection hole 220 is closed, a moving path of the inhalable composition to the reservoir 103 may be blocked, and accordingly, the filling of the reservoir 103 may be stopped.

The operation of opening and closing the injection hole 220 may be controlled according to whether the user presses the lever 300.

As described above, when the lever 300 is pressed, the inclined surface 3301 of the wedge member 330 may push up the bottom surface of the canister 200, that is, a bottom surface of the accommodation portion 210. At this time, the accommodation portion 210 moves in the second direction, but one side of the injection hole 220 may be fixed to the reservoir 103. That is, the position of the injection hole 220 in the housing 101 does not change, but the position of the other side of the injection hole 220 in the accommodation portion 210 may change due to the rising of the accommodation portion 210 in the second direction.

For example, when the other side of the injection hole 220 is pressed deeply into the accommodation portion 210, the injection hole 220 may be opened. Accordingly, the inhalable composition in the accommodation portion 210 may move to the reservoir 103 through the injection hole 220. At this time, the inhalable composition may move by a pressure difference between the reservoir 103 and the accommodation portion 210. That is, when the inhalable composition filled in the reservoir 103 by the user is consumed as the aerosol is discharged, the pressure in the reservoir 103 may be reduced, and therefore, when the injection hole 220 is opened, the inhalable composition may move from the accommodation portion 210 to the reservoir 103. On the other hand, when the reservoir 103 is sufficiently filled with the inhalable composition, the inhalable composition may not additionally move to the reservoir 103 even if the user presses the lever 300 to open the injection hole 220.

Hereinafter, referring to FIGS. 4 and 5, a process in which the lever 300 of the inhaler 10 according to an embodiment operates in two steps to sequentially open the relief valve 700 and the canister 200 and return to its original position will be described.

FIG. 4A shows the inhaler 10 in a state where no external force is applied to the lever 300.

At this time, as shown in FIG. 5A, both the relief valve 700 and the canister 200 are not opened.

FIG. 4B shows the inhaler 10 in a state where the lever 300 is pressed in the first direction and moved to the first position.

At this time, the support member 310 restricts the rotation of the end portion of the protrusion member 320 in a direction away from the canister 200. Accordingly, the protrusion member 320 may come into contact with the other end of the relief bar 800 in a state where the end portion stands up toward the first surface of the housing 101. The relief bar 800 raises in the second direction by the protrusion member 320 to open the relief valve 700. As shown in FIG. 5B, as the relief valve 700 is opened, the composition remaining in the reservoir 103 is discharged to the outside.

FIG. 4C shows the inhaler 10 in a state where the lever 300 is further pressed in the first direction in the state of FIG. 4B and moved to the second position.

As the lever 300 passes the first position, the protrusion member 320 may escape from the contact with the end of the relief bar 800 and the relief bar 800 may return to its original position. That is, the relief bar 800 moves down and the relief valve 700 is closed. In addition, as shown in FIG. 5C, when the lever 300 eventually reaches the second position, the inclined surface of the wedge member 330 raises the accommodation portion 210 of the canister 200 in the second direction, thereby opening the injection hole 220. The inhalable composition may move through the injection hole 220 and the inhalable composition may be smoothly filled in the reservoir 103 from which the residual gas is removed.

FIG. 4D shows the inhaler 10 in a state where the lever 300 has reached the first position while returning to the original position from the second position.

At this time, the inclined surface 3301 moves in a direction opposite to the first direction, and the accommodation portion 210 moves down and the injection hole 220 is closed as shown in FIG. 5D. In other words, the filling of the reservoir 103 with the inhalable composition is complete. The protrusion member 320 may come into contact with the other end of the relief bar 800 at the same time as the contact between the inclined surface 3301 and the bottom surface of the accommodation portion 210 is released. At this time, the support member 310 allows the end portion of the protrusion member 320 to rotate toward the canister 200. Accordingly, the end portion of the protrusion member 320 may not maintain the standing state, but may smoothly pass the other end of the relief bar 800 while rotating toward the canister 200. For example, the protrusion member 320 may avoid the relief bar 800 elastically. As a result, when it moves in a direction opposite to the first direction, the protrusion member 320 does not raise the relief bar 800 in the second direction. Accordingly, the relief valve 700 may remain closed, and the inhalable composition newly filled in the reservoir 103 is not discharged to the outside.

FIG. 4E shows the inhaler 10 in a state where the lever 300 is completely returned to the original position.

As shown in FIG. 5E, the protrusion member 320 which has passed the relief bar 800 may return to the same position as the protrusion member 320 of FIG. 5A. For example, the protrusion member 320 may be provided with an elastic member (not shown) that is compressible in a direction in which the end portion of the protrusion member 320 rotates toward the canister 200. In the operation of 5D, the elastic member may be compressed in a direction in which the end portion of the protrusion member 320 rotates toward the canister 200, and if no external force is applied to the protrusion member 320, the elastic member may rotate the protrusion member 320 to its original position as shown in FIGS. 5A and 5E. Accordingly, when the lever 300 returns to the original position after the filling is finished, the protrusion member 320 may elastically escape the relief bar 800 without pushing it up to return to the original position.

In the inhaler 10 according to an embodiment described above, in order to prevent pressure of a residual gas remaining in the reservoir 103 from hindering a new composition from entering when the reservoir 103 is refilled with the composition using the filling lever 300, the relief valve 700 interlocked with the filling lever 300 may be provided to remove the residual gas in the reservoir 103 before the filling.

In addition, in the inhaler 10 according to an embodiment described above, the filling lever 300 may operate in two steps. In a first step, the relief valve 700 may be opened as a state before the filling, and in a second step, the relief valve 700 may be closed, thereby performing the filling.

While the embodiments of the present disclosure have been described above with reference to specific components, and limited embodiments and drawings, the above descriptions are merely for better understanding of the present disclosure, and it will be apparent to one of ordinary skill in the art that various changes in form and details may be made in these embodiments without departing from the spirit and scope of the claims and their equivalents. For example, suitable results may be achieved if the described techniques are performed in a different order and/or if components in a described system, architecture, device, or circuit are combined in a different manner and/or replaced or supplemented by other components or their equivalents. Accordingly, the scope of the present disclosure is defined not by the detailed description, but by the claims and their equivalents, and all variations within the scope of the claims and their equivalents are to be construed as being included in the disclosure.

1. An inhaler comprising:
a housing having one surface, the other surface opposite to the one surface, and a plurality of side surfaces connecting the one surface and the other surface;
a reservoir which is disposed inside the housing and stores an inhalable composition;
a canister which accommodates the inhalable composition to be stored in the reservoir and is connected to the reservoir to be opened or closed;
a relief valve which is disposed to be spaced apart from the canister and provided to open and close the reservoir to the outside; and
a lever which controls an opening and closing operation of at least one of the canister or the relief valve,
wherein the lever controls the canister to be opened after the relief valve is opened.

2. The inhaler of claim 1, further comprising:
a relief bar having one end coupled to the relief valve and extending toward the lever,
wherein the relief bar is positioned between the lever and the canister.

3. The inhaler of claim 2,
wherein the lever further comprises:
a support member which extends in a first direction toward the canister; and
a protrusion member having an end portion rotatably coupled to the support member around a rotation shaft provided in the support member, and
wherein the other end of the relief bar is positioned between the protrusion member and the other surface of the housing.

4. The inhaler of claim 3, wherein, when the lever is pressed, the protrusion member moves the relief bar in a second direction toward the one surface of the housing while being moved in the first direction.

5. The inhaler of claim 3,
wherein, when the lever is pressed in the first direction, the support member restricts rotation of the protrusion member, and
wherein, when the lever returns to an original position, the support member allows the rotation of the protrusion member.

6. The inhaler of claim 3, wherein the protrusion member is disposed so that rotation in a direction toward the canister is not restrained and rotation in a direction away from the canister is restrained in a state where the end portion stands toward the one surface of the housing.

7. The inhaler of claim 3, wherein, when the canister is opened, the protrusion member is positioned between the relief bar and the canister.

8. The inhaler of claim 3,
wherein the lever further comprises a wedge member extending toward a bottom surface of the canister, and
wherein a distance between the wedge member and the bottom surface of the canister is greater than a distance between the protrusion member and the relief bar.

9. The inhaler of claim 8,
wherein the wedge member comprises an inclined surface inclined to be adjacent to the bottom surface along the first direction, and
wherein, when the lever is further pressed in the first direction in a state where the inclined surface comes into contact with the bottom surface, the inclined surface moves the canister in the second direction toward the one surface of the housing.

10. The inhaler of claim 1,
wherein, when the lever reaches a first position in which the relief valve is opened, the inhalable composition remaining in the reservoir is discharged to the outside, and
wherein, when the lever reaches a second position in which one side of the canister is opened, the reservoir is filled with the inhalable composition accommodated in the canister.

11. The inhaler of claim 2, wherein the relief bar opens the relief valve when the relief bar moves in a second direction toward the one surface of the housing, and closes the relief valve when the relief bar returns to an original position.

12. The inhaler of claim 1,
wherein the canister comprises:
an accommodation portion which accommodates the inhalable composition; and
an injection hole having one side connected to the reservoir and the other side positioned in the accommodation portion, and
wherein the injection hole is opened or closed to allow or restrict movement of the inhalable composition to the reservoir.

13. The inhaler of claim 12, wherein the injection hole is opened when the accommodation portion moves in a second direction from the other surface to the one surface of the housing, and is closed when the accommodation portion returns to an original position.
